(19) United States
(12) Patent Application Publication
(10) Pub. No.: US 2004/0057915 A1
(43) Pub. Date: Mar. 25, 2004

(54) Cosmetic or dermatological formulations that are visible on the skin and uses for same

(75) Inventors: Heinrich Gers-Barlag, Kummerfeld (DE); Anja Muller, Rumpel (DE); Birgit Groteluschen, Hamburg (DE); Wiebke Grundt, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/341,935

(22) Filed: Jan. 14, 2003

Related U.S. Application Data
(63) Continuation of application No. PCT/EP01/07983, filed on Jul. 11, 2001.

(30) Foreign Application Priority Data
Jul. 14, 2000 (DE) 100 34 332.5

(51) Int. Cl. A61K 7/42; A61K 7/021

(52) U.S. Cl. 424/59; 424/63

(57) ABSTRACT

The invention is a cosmetic or dermatological formulation and a method of applying same. The cosmetic or dermatological formulation includes one or more effect pigments, particularly pearlescent pigments, having an average particle size of from 15 μm to 200 μm for making the cosmetic or dermatological formulation visible on the skin. The invention also includes a method of assessing the amount of a cosmetic and dermatological formulation applied to the skin.

### CROSS-REFERENCE TO RELATED APPLICATIONS

This is a continuation application of PCT/EP01/07983, filed Jul. 11, 2001, which is incorporated herein by reference in its entirety, and also claims the benefit of German Priority Application No. 100 34 332.5, filed Jul. 14, 2000.

### FIELD OF THE INVENTION

The present invention relates to the use of pigments for making cosmetic and dermatological formulations, in particular cosmetic and dermatological light protection formulations, visible on the skin.

### BACKGROUND OF THE INVENTION

In addition to the positive effects of sunlight, such as general well-being, the formation of vitamin D3 and the treatment of acne, there are also negative effects which must be counteracted.

If the skin is exposed for too long to the sun or to a source of artificial rays, after a latency period of from 2 to 3 hours, a reddening of the skin, starkly demarcated from the unradiated skin, arises (solar erythema). Sunburn which has arisen in this way is differentiated as follows:

1st degree: erythema (reddening, feeling of warmth)

subsides again after 2 to 3 days and disappears with a simultaneous increase in pigmentation,

2nd degree: blister formation

blisters form on the skin with burning and itching, and sections of the epidermis are shed

3rd degree: cell damage

deep cell damage occurs, the body reacts with fever, large sections of the epidermis are shed.

The 2nd and 3rd degrees are also referred to as solar dermatitis.

The formation of the erythema is dependent on the wavelength. The erythema region of UV-B is between 280 nm and 320 nm.

Approximately 90% of the ultraviolet radiation that reaches the Earth consists of UV-A rays with a wavelength between 320 nm and 400 nm. Whereas UV-B radiation varies widely depending on numerous factors (e.g. season and time of day or latitude), UV-A radiation remains relatively constant from day to day irrespective of seasonal and diurnal or geographic factors. At the same time, most of the UV-A radiation penetrates into the living epidermis, while about 70% of the UV-B rays are retained by the horny layer.

For a long time it has incorrectly been assumed that the long-wavelength UV-A radiation has only a negligible biological effect and that, correspondingly, the UV-B rays are responsible for most photodamage to the human skin. In the meantime, however, numerous studies have proven that UV-A radiation is far more hazardous than UV-B radiation with regard to the triggering of photodynamic, specifically phototoxic, reactions and chronic changes in the skin. The harmful effect of UV-B radiation can also be intensified by UV-A radiation.

Sunburn or photoerythema are the acute manifestations of the effect of light. Moreover, however, on areas of skin that are exposed to a particularly high degree (face, neck, hands), an actinic change in the skin caused by radiation arises with increasing age due to the high overall dose connected therewith. The most common chronic photodamage to the skin is actinic and senile elastosis. Macroscopically, it manifests itself in a thickening and coarsening of the skin, wrinkling, loss of elasticity, appearance of yellowish deposits that show through the skin and irregular pigment accumulations. The epidermis becomes thin in places and exhibits warty growths, the dermis loses its elasticity and tension, and the water-binding capacity is reduced. Chronic photodamage, which subsequently arises, also includes malignant melanoma and in the last stage, actinic keratosis.

Since the contributions of the various wavelength ranges of UV light have still not been fully explained, preventative protection both for the UV-A region and also the UV-B region is of decisive importance, for example by applying light protection filter substances in the form of a cosmetic or dermatological formulation to the skin. Cosmetic preparations should in principle absorb the critical UV-A rays to a high degree, not only to protect sensitive skin during sunbathing, but also for general protection ranging to use in a normal skin cream since skin aging and the risk of skin cancer are significantly influenced by this part of UV light.

The purpose of a sunscreen preparation is to prolong the time for which a consumer can remain in the sun without suffering photodamage. In this connection, the filter effect, particularly for the UV-B region, should be adapted to the individual sensitivity of the consumer and to the intensity of the solar irradiation.

The weakening of the intensity of the irradiated light and thus the effectiveness of a sunscreen formulation are dependent, inter alia, on the layer thickness of the applied filter substance. As the layer thickness increases, so too does the effectiveness of a sunscreen composition. It is therefore only possible to a limited extent to increase the sun protection factor by increasing the concentration of the UV filter substance. Particularly in the case of low-viscosity preparations, such as oils or alcoholic solutions, it is, moreover, necessary, above a certain limiting concentration of filter substance, to also increase the viscosity of the formulation and thus the layer thickness that can be achieved on the skin.

The layer thickness achieved with a special base is a very important criterion in light protection. In general, the sun protection factor increases in the order: oil<alcoholic solution<liquid emulsion<cream<paste. A further important parameter in this connection is also the adhesion capacity and water resistance of the formulations on the skin. Most sunscreen compositions are applied in the proximity of water or during sporting activity (perspiration). A water resistant sunscreen composition can protect the user from sunburn not only during bathing, but also after bathing provided it is not washed off to too great a degree.

The prior art of course recognizes a large number of different light protection formulations with a very wide variety of sun protection factors and application properties. A disadvantage of these customary sunscreen compositions is that the user, after rubbing in the cream, can neither recognize whether he has covered all of the body parts exposed to radiation with a protecting layer, nor whether coverage of the skin is adequate, i.e., whether the layer thickness chosen is sufficient. A further disadvantage is that the user can not monitor how the layer thickness of the sunscreen composition changes, for example as a result of contact with water or mechanical stresses, such as drying, etc., and when, accordingly, a renewed rubbing in of the cream has become necessary.

In the worst case, this may lead to a reduced protective action of the products.

### SUMMARY OF THE INVENTION

Accordingly, it was an object of the present invention to overcome the disadvantages of the prior art and find ways of visualizing cosmetic and dermatological formulations, in particular cosmetic and dermatological sunscreen formulations, on the skin, thus facilitating application in a uniform film on the skin.

It was surprising and could in no way be foreseen by the person skilled in the art that

the use of effect pigments having an average particle size of from 15 μm to 150 μm for making cosmetic and dermatological formulations visible on the skin

would overcome the disadvantages of the prior art.

Although the prior art recognizes light protection preparations with a content of pigments, the pigments serve as so-called physical UV filters which reflect the incident light and thus develop a light protection effect. However, preparations of this type are formulated so that they are transparent and are not visible on the skin since so-called whitening (the physical filters are usually inorganic metal oxides) is undesired from a cosmetics viewpoint and not accepted by the consumer. Accordingly, the prior art was unable to point the way to the present invention.

### DETAILED DESCRIPTION OF THE INVENTION

For the purposes of the present invention, effect pigments are understood as meaning those pigments which, as a result of light reflections, in particular under solar irradiation, lead to a glittering optical effect (shimmering effect).

Advantageous effect pigments for the purposes of the present invention are, for example, pearlescent pigments. Preference is given in particular to the types of pearlescent pigments listed below:

1. Natural pearlescent pigments, such as, for example

“pearl essence” (guanine/hypoxanthine mixed crystals from fish scales) and

“mother of pearl” (ground mussel shells)

2. Monocrystalline pearlescent pigments, such as, for example, bismuth oxychloride (BiOCl)

3. Layer substrate pigments: e.g. mica/metal oxide

Bases for pearlescent pigments are, for example, pulverulent pigments or castor oil dispersions of bismuth oxychloride and/or titanium dioxide, and bismuth oxychloride and/or titanium dioxide on mica. Such pigments are also advantageous for the purposes of the present invention. Particular preference is given, for example, to the luster pigment listed under CIN 77163.

Also advantageous are, for example, the following types of pearlescent pigment based on mica/metal oxide:

| Group | Coating/layer | Color |
|---|---|---|
| Silver-white pearlescent | TiO₂: 40-60 nm | silver |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color luster pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Berlin blue | deep blue |
| | TiO₂/Carmine | red |

Particular preference is given, for example, to the pearlescent pigments obtainable from Merck under the trade names Timiron, Colorona or Dichrona.

The list of said pearlescent pigments is not of course intended to be limiting. Pearlescent pigments which are advantageous for the purposes of the present invention are obtainable by numerous methods known per se. For example, substrates other than mica can be coated with further metal oxides, such as, for example, silica and the like. For example, SiO₂ particles coated with TiO₂ and Fe₂O₃ (“ronaspheres”), which are sold by Merck, are advantageous.

It may, moreover, be advantageous to dispense entirely with a substrate such as mica. Particular preference is given to iron pearlescent pigments prepared without the use of mica. Such pigments are obtainable, for example, under the trade name Sicopearl Kupfer 1000 from BASF.

It may be further advantageous to use pigments from Engelhard/Mearl which are based on calcium sodium borosilicate coated with titanium dioxide. These are available under the trade name Reflecks. In addition to the color, they have a glitter effect as a result of their particle size from 40 to 180 μm. Also particularly advantageous are effect pigments which are available under the trade name Metasomes Standard/Glitter in different colors (yellow, red, green, blue) from Flora Tech. The glitter particles are present here in mixtures with various auxiliaries and dyes (such as, for example, the dyes with the Colour Index (CI) numbers 19140, 77007, 77289, 77491).

The effect pigments according to the invention advantageously have an average particle size of from 15 μm to 200 μm, particularly advantageously 50 μm to 150 μm, in particular 100 μm to 150 μm.

The effect pigments can either be present individually or else as a mixture, and mutually coated with one another, different coating thicknesses generally resulting in different color effects. The total amount of the pigments is advantageously chosen from the range from, for example, 0.1% by weight to 30% by weight, preferably from 0.5 to 15% by weight, in particular from 1.0 to 10% by weight, in each case based on the total weight of the preparations.

For the purposes of the present invention, the cosmetic or dermatological formulations can additionally comprise one or more water phases as well as one or more oil phases and, for example, be in the form of W/O, O/W, W/O/W, O/W/O or other multiple emulsions. Such emulsions may preferably also be a microemulsion, a Pickering emulsion or a sprayable emulsion.

The preparations for the purposes of the present invention can be formulated as liquid, pasty or solid preparations, for example as aqueous or alcoholic solutions, aqueous suspensions, hydrodispersions, oleogels, ointments, creams, gels, oils, powders or sticks. Depending on the desired formulation, active ingredients can be incorporated into the bases which comprise, as further components, for example, oil components, fats and waxes, emulsifiers, anionic, cationic, ampholytic, zwitterionic and/or nonionogenic surfactants, lower mono- and polyhydric alcohols, water, preservatives, buffer substances, thickeners, fragrances, dyes and opacifiers.

The cosmetic and/or dermatological formulations for the purposes of the present invention can comprise the ingredients customary for the respective form and be used for cosmetic and/or dermatological light protection, and also for the treatment, care and cleansing of the skin and/or of the hair. Accordingly, for the purposes of the present invention, cosmetic or topical dermatological compositions can, depending on their make-up, be used, for example, as skin protection cream, cleansing milk, sunscreen lotion, nourishing cream, day or night cream, etc. It is likewise possible and advantageous to use the compositions according to the invention as a base for pharmaceutical formulations.

For use, the cosmetic and dermatological preparations according to the invention are applied in an adequate amount to the skin and/or the hair in the manner customary for cosmetics.

The cosmetic and dermatological preparations according to the invention can also comprise cosmetic auxiliaries, as are customarily used in such preparations, e.g. preservatives, bactericides, perfumes, antifoams, dyes, pigments which have a coloring action, emollients, moisturizing substances and/or humectants or other customary constituents of a cosmetic or dermatological formulation.

According to the invention, the preparations advantageously further comprise one or more antioxidants. Antioxidants which are favorable, but nevertheless optional, are all antioxidants which are suitable or customary for cosmetic and/or dermatological applications. It is advantageous to use antioxidants as the sole active ingredient class when, for example, a cosmetic or dermatological application is at the fore, such as, for example, the control of oxidative stress of the skin. It is, however, also favorable to provide the novel preparations with a content of one or more antioxidants if the intention is for the preparations to serve another purpose, e.g. as sunscreen compositions.

The antioxidants are particularly advantageously chosen from the group consisting of amino acids (e.g. glycine, histidine, tyrosine, tryptophan) and derivatives thereof, imidazoles, (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximines) in very low tolerated doses (e.g. pmol to μmol/kg), and also (metal) chelating agents (e.g. α-hydroxyfatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and derivatives thereof, α-glycosylrutin, ferulic acid, furfurylideneglucitol, carnosine, butylated hydroxytoluene, butylated hydroxyanisole, nordihydroguaiac acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide), and the derivatives (salts, esters, ethers, sugars, nucleotides, nucleosides, peptides and lipids) of said active substances which are suitable according to the invention.

The amount of the abovementioned antioxidants (one or more compounds) in the preparations according to the invention is preferably from 0.001 to 30% by weight, particularly preferably from 0.05 to 20% by weight, in particular from 0.1 to 10% by weight, based on the total weight of the preparation.

If vitamin E and/or derivatives thereof are used as the antioxidant or antioxidants, their respective concentrations are advantageously chosen from the range from 0.001 to 10% by weight, based on the total weight of the formulation.

If vitamin A or vitamin A derivatives or carotenes or derivatives thereof are used as the antioxidant or antioxidants, their respective concentrations are advantageously chosen from the range from 0.001 to 10% by weight, based on the total weight of the formulation.

In particular, cosmetic and dermatological preparations which are in the form of a sunscreen composition are favorable. In addition to the pigments used according to the invention, these preferably additionally comprise at least one UV-A filter substance and/or at least one UV-B filter substance. Such formulations may, but do not have to, optionally comprise further inorganic pigments as UV filter substances.

Preference is given to inorganic pigments based on metal oxides and/or other metal compounds which are insoluble or sparingly soluble in water, in particular the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides.

An additional content of stabilizing titanium dioxide and/or zinc oxide particles may also of course be advantageous, but is not necessary for the purposes of the present invention.

It is also advantageous for the purposes of the present invention to provide those cosmetic and dermatological preparations whose main purpose is not protection against sunlight, but which nevertheless have a content of UV protection substances. Thus, for example, UV-A and/or UV-B filter substances are often incorporated into day creams.

UV protection substances, like antioxidants and, if desired, preservatives, also represent effective protection of the preparations themselves against decay. Preparations according to the invention advantageously comprise substances which absorb UV radiation in the UV-A and UV-B region, the total amount of filter substances being, for example, from 0.1% by weight to 30% by weight, preferably from 0.5 to 20% by weight, in particular from 1.0 to 15.0% by weight, based on the total weight of the preparations, in order to provide cosmetic preparations which protect the hair or the skin from the whole range of ultraviolet radiation. They can also be used as sunscreens for the hair or the skin.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

Further advantageous UV-A filter substances are phenylene-1,4-bis (2-benzimidazyl)-3,3′-5,5′-tetrasulfonic acid:

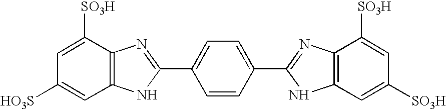

and its salts, particularly the corresponding sodium, potassium or triethanolammonium salts, in particular phenylene-1,4-bis(2-benzimidazyl)-3,3′-5,5′-tetrasulfonic bis-sodium salt:

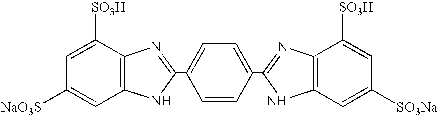

and 1,4-di(2-oxo-10-sulfo-3-bornylidenemethyl)benzene and salts thereof (in particular the corresponding 10-sulfato compounds, in particular the corresponding sodium, potassium or triethanolammonium salt), which is also referred to as benzene-1,4-di(2-oxo-3-bornylidenemethyl-10-sulfonic acid) and is characterized by the following structure:

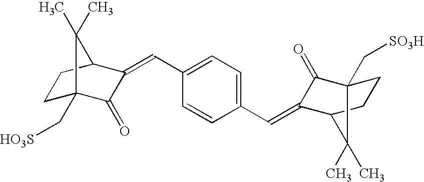

Advantageous UV filter substances for the purposes of the present invention are also broad-band filters, i.e. filter substances which absorb both UV-A and UV-B radiation.

Advantageous broad-band filters and/or UV-B filter substances are, for example, bisresorcinyltriazine derivates having the following structure:

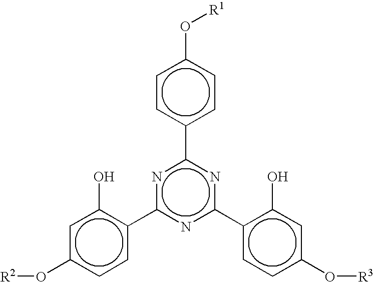

where R¹, R² and R³ independently of one another are chosen from the group of branched and unbranched alkyl groups having 1 to 10 carbon atoms, or are a single hydrogen atom. Particular preference is given to 2,4-bis{[4-(2-ethylhexyloxy)-2-hydroxy]phenyl}-6-(4-methoxyphenyl )-1, 3,5-triazine (INCI: Aniso Triazine), which is available under the trade name Tinosorb® S from CIBA-Chemikalien GmbH, and to tris(2-ethylhexyl) 4,4′, 4″-(1,3,5-triazine-2,4,6-triyltriimino)trisbenzoate, synonym: 2,4,6-tris[anilino(p-carbo-2′-ethyl-1′-hexyloxy)]-1,3,5-triazine (INCI: Octyl Triazone), which is marketed by BASF Aktiengesellschaft under the trade name UVINUL® T 150.

Other UV filter substances which have the structural formula

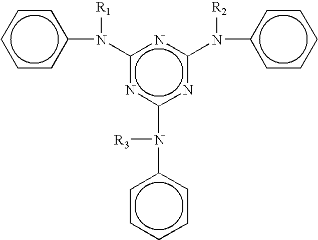

are also advantageous UV filter substances for the purposes of the present invention, for example the s-triazine derivatives described in European Laid-Open Specification EP 570 838 A1, whose chemical structure is given by the generic formula

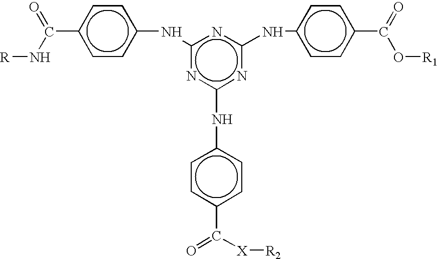

where

R is a branched or unbranched C₁-C₁₈-alkyl radical, a C₅-C₁₂-cycloalkyl radical, optionally substituted with one or more C₁-C₄-alkyl groups,

X is an oxygen atom or an NH group,

R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

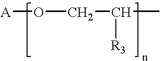

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

R₂ is a branched or unbranched C₁-C₁₈-alkyl radical, a C₅-C₁₂-cycloalkyl radical, optionally substituted by one or more C₁-C₄-alkyl groups, when X is the NH group, and

a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

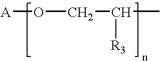

in which

A is a branched or unbranched C₁-C₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

when X is an oxygen atom.

A particularly preferred UV filter substance for the purposes of the present invention is also an asymmetrically substituted s-triazine, the chemical structure of which is given by the formula

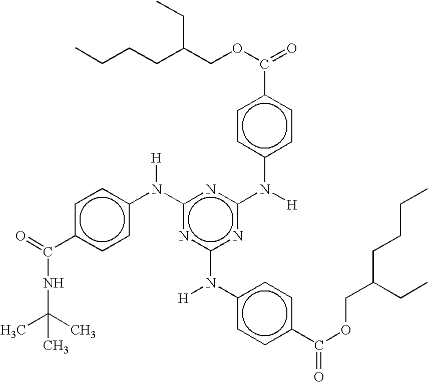

which is also referred to below as dioctylbutylamidotriazone (INCI: Dioctylbutamidotriazone) and is available under the trade name UVASORB HEB from Sigma 3V.

European Laid-Open Specification 775 698 also describes preferred bisresorcinyltriazine derivatives, the chemical structure of which is given by the generic formula

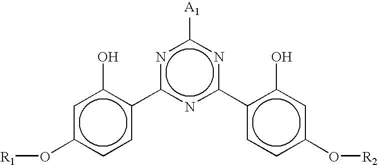

where R₁, R₂ and A₁ represent very different organic radicals.

Also advantageous for the purposes of the present invention are 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis{[4-tris(trimethyl-siloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

An advantageous broad-band filter for the purposes of the present invention is 2,2′-methylenebis(6-(2H-benzotriazol-2-yl)-4-( 1,1,3,3-tetramethylbutyl)phenol), which is characterized by the chemical structural formula

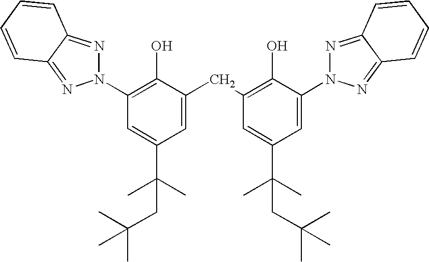

and is available under the trade name Tinosorb®) M from CIBA-Chemikalien GmbH.

Another advantageous broad-band filter for the purposes of the present invention is 2-(2H-benzotriazol-2-yl )-4-methyl-6-[2-methyl-3-[1,3,3,3-tetramethyl-1-[(trimethylsilyl)oxy]disiloxanyl]propyl]phenol (CAS No.: 155633-54-8) having the INCI name Drometrizole Trisiloxane, which is characterized by the chemical structural formula

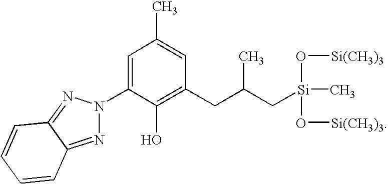

The UV-B filters can be oil-soluble or water-soluble. Examples of advantageous oil-soluble UV-B filter substances are:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate;

2,4,6-trianilino(p-carbo-2′-ethyl-1′-hexyloxy)-1,3,5-triazine;

esters of benzalmalonic acid, preferably di(2-ethylhexyl) 4-methoxybenzalmalonate;

esters of cinnamic acid, preferably 2-ethylhexyl 4-methoxycinnamate, isopentyl 4-methoxycinnamate;

derivates of benzophenone, preferably 2-hydroxy-4-methoxybenzophenone, 2-hydroxy-4-methoxy-4′-methylbenzophenone, 2,2′-dihydroxy-4-methoxybenzophenone

and UV filters bonded to polymers.

Examples of advantageous water-soluble UV-B filter substances are:

salts of 2-phenylbenzimidazole-5-sulfonic acid, such as its sodium, potassium or its triethanolammonium salt, and also the sulfonic acid itself;

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

A further light protection filter substance which can be used advantageously according to the invention is ethylhexyl 2-cyano-3,3-diphenylacrylate(octocrylene), which is available from BASF under the name Uvinul® N 539 and is characterized by the following structure:

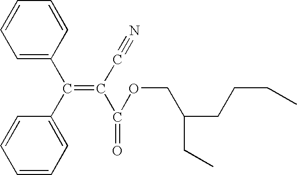

It can also be of considerable advantage to use polymer-bonded or polymeric UV filter substances in the preparations according to the present invention, in particular those described in WO-A-92/20690.

In some instances, it can also be advantageous to incorporate further UV-A and/or UV-B filters in accordance with the invention into cosmetic or dermatological preparations, for example certain salicylic acid derivatives, such as 4-isopropylbenzyl salicylate, 2-ethylhexyl salicylate (=octyl salicylate), homomenthyl salicylate.

The list of given UV filters which can be used for the purposes of the present invention is not of course intended to be limiting.

The invention further provides a cosmetic method of assessing the amount of cosmetic and dermatological formulations applied to the skin, which comprises applying effect pigments having an average particle size of from 15 μm to 200 μm, which are incorporated into a cosmetic preparation, to the skin and, by reference to the visible effect, estimating on which areas of the skin application has already taken place and/or whether sufficient preparation has been applied and/or when renewed application has become necessary.

The examples below serve to illustrate the present invention without limiting it. The numerical values in the examples are percentages by weight, based on the total weight of the respective preparations.

% by weight

Example 1

| | |
|---|---|
| O/W lotion | |
| Glyceryl stearate SE | 3.50 |
| Stearic acid | 1.80 |
| Glycerol | 3.00 |
| Cetylstearyl alcohol | 0.50 |
| Octyldodecanol | 7.00 |
| Dicaprylyl ether | 8.00 |
| Tris(2-ethylhexyl) 4,4′,4″-(1,3,5-triazine-2,4,6-triyltri- | 3.00 |
| imino)trisbenzoate | |
| Aerosil ® | 1.00 |
| Effect pigment | 4.00 |
| Carbomer | 0.20 |
| Sodium hydroxide (45% strength) | 0.20 |
| Preservative | q.s. |
| Perfume | q.s. |
| Water, demin. | ad 100.00 |

Example 2

| | |
|---|---|
| O/W lotion | |
| Glyceryl stearate SE | 3.00 |
| Stearic acid | 2.00 |
| Glycerol | 10.00 |
| Cetylstearyl alcohol | 0.50 |
| Octyldodecanol | 6.00 |
| Dicaprylyl ether | 8.00 |
| Tris(2-ethylhexyl) 4,4′,4″-(1,3,5-triazine-2,4,6-triyltri- | 5.00 |
| imino)trisbenzoate | |
| Aerosil ® | 0.50 |
| Effect pigment | 6.00 |
| Carbomer | 0.20 |
| Sodium hydroxide (45% strength) | 0.20 |
| Preservative | q.s. |
| Perfume | q.s. |
| Water, demin. | ad 100.00 |

Example 3

| | |
|---|---|
| Hydrodispersion gel | |
| Pemulen ® TR-1 | 0.50 |
| Ethanol | 3.50 |
| Glycerol | 3.00 |
| Dimethicone | 1.50 |
| Octyldodecanol | 0.50 |
| Caprylic/capric triglyceride | 5.00 |
| Effect pigment | 7.00 |
| Tris(2-ethylhexyl) 4,4′,4″-(1,3,5-triazine-2,4,6-triyltri- | 5.00 |
| imino)trisbenzoate | |
| Carbomer | 0.20 |
| Sodium hydroxide (45% strength) | 0.55 |
| Preservative | q.s. |
| Perfume | q.s. |
| Water, demin. | ad 100.00 |

Example 4

| | |
|---|---|
| Emulsifier-free sun lotion SPF 30 | |
| Caprylic/capric triglycerides | 30.00 |
| Tris(2-ethylhexyl) 4,4′,4″-(1,3,5-triazine-2,4,6-triyltri- | 4.00 |
| imino)trisbenzoate | |
| Methylbenzylidene camphor | 2.00 |
| Ethylhexyl salicylate | 5.00 |
| Butylmethoxydibenzoylmethane | 2.00 |
| Eusolex T2000 | 4.00 |
| Aerosil ® R972 | 2.00 |
| Effect pigment | 5.00 |
| Zinc oxide neutral | 2.50 |
| Cetylhydroxyethylcellulose | 0.50 |
| Glycerol | 10.00 |
| Water | ad 100.00 |

Example 5

| | |
|---|---|
| O/W cream | |
| Glyceryl stearate SE | 3.50 |
| Stearic acid | 3.50 |
| Butylene glycol | 5.00 |
| Cetylstearyl alcohol | 3.00 |
| C₁₂-C₁₅-alkyl benzoates | 10.00 |
| Dioctylbutylamidotriazone | 4.00 |
| Aerosil ® | 3.00 |
| Effect pigment | 10.00 |
| Carbomer | 0.20 |
| Sodium hydroxide (45% strength) | 0.35 |
| Preservative | q.s. |
| Perfume | q.s. |
| Water, demin. | ad 100.00 |

Example 6

| | |
|---|---|
| W/O lotion | |
| Polyglyceryl-2 polyhydroxystearate | 3.50 |
| Polyglyceryl-3 diisostearate | 3.50 |
| Butylene glycol | 5.00 |
| Ceresine | 3.00 |
| C₁₂-C₁₅-alkyl benzoates | 10.00 |
| Tris(2-ethylhexyl) 4 ,4′,4″-(1,3,5-triazine-2,4,6-triyltri- | 4.00 |
| imino)trisbenzoate | |
| Effect pigment | 8.00 |
| Vaseline | 2.00 |
| Sodium hydroxide (45% strength) | 0.35 |
| Preservative | q.s. |
| Perfume | q.s. |
| Water, demin. | ad 100.00 |

Example 7

| | |
|---|---|
| O/W lotion | |
| Glyceryl stearate SE | 3.50 |
| Stearic acid | 1.80 |
| Glycerol | 3.00 |
| Cetylstearyl alcohol | 0.50 |
| Octyldodecanol | 7.00 |
| Dicaprylyl ether | 8.00 |
| Tris(2-ethylhexyl) 4,4′,4″-(1,3,5-triazine-2,4,6-triyltri- | 3.00 |
| imino)trisbenzoate | |
| Butylmethoxydibenzoylmethane | 2.00 |
| Methylbenzylidenecamphor | 1.00 |
| TiO₂ | 2.00 |
| Effect pigment | 5.00 |
| Carbomer | 0.20 |
| Sodium hydroxide (45% strength) | 0.20 |
| Preservative | q.s. |
| Perfume | q.s. |
| Water, demin. | ad 100.00 |

Example 8

| | |
|---|---|
| Hydrodispersion gel | |
| Acrylate/C₁₀₋₃₀-alkyl acrylate crosspolymer | 0.50 |
| Ethanol | 3.50 |
| Glycerol | 3.00 |
| Dimethicone | 1.50 |
| Octyldodecanol | 0.50 |
| Caprylic/capric triglyceride | 5.00 |
| Aerosil ® | 2.00 |
| 2,4-Bis{[4-(2-ethylhexyloxy)-2-hydroxy]phenyl}-6-(4- | 5.00 |
| methoxyphenyl)-1,3,5-triazine | |
| Butylmethoxydibenzoylmethane | 2.00 |
| Methylbenzylidenecamphor | 1.00 |
| Effect pigment | 9.00 |
| Carbomer | 0.20 |
| Sodium hydroxide (45% strength) | 0.55 |
| Preservative | q.s. |
| Perfume | q.s. |
| Water, demin. | ad 100.00 |

Example 9

| | |
|---|---|
| O/W cream | |
| Glyceryl stearate SE | 3.50 |
| Stearic acid | 3.50 |
| Butylene glycol | 5.00 |
| Cetylstearyl alcohol | 3.00 |
| C₁₂-C₁₅-alkyl benzoates | 10.00 |
| Uvinul ® T150 | 4.00 |
| Butylmethoxydibenzoylmethane | 2.00 |
| Methylbenzylidenecamphor | 1.00 |
| Effect pigment | 4.00 |
| Carbomer | 0.20 |
| Sodium hydroxide (45% strength) | 0.35 |
| Preservative | q.s. |
| Perfume | q.s. |
| Water, demin. | ad 100.00 |

Example 10

| | |
|---|---|
| W/O lotion | |
| Polyglyceryl-2 polyhydroxystearate | 3.50 |
| Polyglyceryl-3 diisostearate | 3.50 |
| Butylene glycol | 5.00 |
| Ceresine | 3.00 |
| C₁₂-C₁₅-alkyl benzoates | 10.00 |
| Triazine | 4.00 |
| Effect pigment | 7.00 |
| Butylmethoxydibenzoylmethane | 2.00 |
| Methylbenzylidenecamphor | 1.00 |
| TiO₂ | 2.00 |
| Vaseline | 2.00 |
| Sodium hydroxide (45% strength) | 0.35 |
| Preservative | q.s. |
| Perfume | q.s. |
| Water, demin. | ad 100.00 |

Example 11

| | |
|---|---|
| O/W lotion | |
| Glyceryl stearate SE | 3.00 |
| Stearic acid | 2.00 |
| Glycerol | 10.00 |
| Cetylstearyl alcohol | 0.50 |
| Octyldodecanol | 7.00 |
| Dicaprylyl ether | 8.00 |
| Tris(2-ethylhexyl) 4,4′,4″-(1,3,5-triazine-2,4,6-triyltri- | 3.00 |
| imino)trisbenzoate | |
| Butylmethoxydibenzoylmethane | 2.00 |
| Aerosil ® | 1.00 |
| Metasomes Standard/Glitter Blue | 1.00 |
| Carbomer | 0.20 |
| Sodium hydroxide (45% strength) | 0.20 |
| Preservative | q.s. |
| Perfume | q.s. |
| Water, demin. | ad 100.00 |

Example 12

| | |
|---|---|
| Hydrodispersion gel | |
| Pemulen ® TR-1 | 0.50 |
| Ethanol | 3.50 |
| Glycerol | 8.00 |
| Dimethicone | 1.50 |
| Octyldodecanol | 0.50 |
| Caprylic/capric triglyceride | 5.00 |
| Metasomes Standard/Glitter Yellow | 5.00 |
| Tris(2-ethylhexyl) 4,4′,4″-(1,3,5-triazine-2,4,6-triyltri- | 5.00 |
| imino)trisbenzoate | |
| Methylbenzylidenecamphor | 4.00 |
| Octocrylene | 7.50 |
| Carbomer | 0.20 |
| Sodium hydroxide (45% strength) | 0.55 |
| Preservative | q.s. |
| Perfume | q.s. |
| Water, demin. | ad 100.00 |

Example 13

| | |
|---|---|
| Emulsifier-free sun lotion SPF 30 | |
| Caprylic/capric triglyceride | 30.00 |
| Tris(2-ethylhexyl) 4,4′,4″-(1,3,5-triazine-2,4,6-triyltri- | 4.00 |
| imino)trisbenzoate | |
| Methylbenzylidenecamphor | 2.00 |
| Ethylhexyl salicylate | 5.00 |
| Butylmethoxydibenzoylmethane | 2.00 |
| Eusolex T2000 | 4.00 |
| Aerosil ® R972 | 2.00 |
| Metasomes Standard/Glitter Green | 1.50 |
| Zinc oxide neutral | 2.50 |
| Cetylhydroxyethylcellulose | 0.50 |
| Glycerol | 10.00 |
| Water | ad 100.00 |

Example 14

| | |
|---|---|
| W/O lotion | |
| Polyglyceryl-2 polyhydroxystearate | 4.50 |
| Polyglyceryl-3 diisostearate | 3.00 |
| Butylene glycol | 5.00 |
| Ceresine | 3.00 |
| C₁₂-C₁₅-alkyl benzoates | 10.00 |
| Tris(2-ethylhexyl) 4,4′,4″-(1,3,5-triazine-2,4,6-triyltri- | 4.00 |
| imino)trisbenzoate | |
| Titanium dioxide | 3.00 |
| Metasomes Standard/Glitter Red | 4.00 |
| Vaseline | 2.00 |
| Sodium hydroxide (45% strength) | 0.35 |
| Preservative | q.s. |
| Perfume | q.s. |
| Water, demin. | ad 100.00 |

That which is claimed:

1. A cosmetic or dermatological formulation, comprising one or more effect pigments having an average particle size of from 15 μm to 200 μm to make the cosmetic and dermatological formulation visible on the skin.

2. The formulation as claimed in claim 1, wherein the average particle size of the one or more effect pigments is in the range from 50 μm to 150 μm.

3. The formulation as claimed in claim 1, wherein the one or more effect pigments are selected from the group consisting of pearlescent pigments.

4. The formulation as claimed in claim 3, wherein the one or more effect pigments are selected from the group consisting of natural pearlescent pigments.

5. The formulation as claimed in claim 4, wherein the one or more effect pigments are selected from the group consisting of pearl essence and mother of pearl.

6. The formulation as claimed in claim 1, wherein the total amount of the one or more effect pigments is in the range from 0.1% to 30% by weight, based on the total weight of the formulation.

7. The formulation as claimed in claim 1, wherein the cosmetic or dermatological formulation comprises at least one substance that absorbs UV radiation in the UV-A or UV-B region.

8. The formulation as claimed in claim 1, wherein the cosmetic or dermatological formulation comprises additional inorganic pigments that serve as UV filter substances.

9. A method for making a cosmetic or dermatological formulation visible on the skin, comprising the step of applying a cosmetic or dermatological formulation to the skin that includes one or more effect pigments having an average particle size of from 15 μm to 200 μm.

10. The method as claimed in claim 9, wherein said applying step further comprises applying the cosmetic or dermatological formulation to provide light protection to the skin.

11. The method as claimed in claim 10, wherein the cosmetic or dermatological formulation comprises at least one substance that absorbs UV radiation in the UV-A or UV-B region.

12. The method as claimed in claim 10, wherein the cosmetic or dermatological formulation comprises additional inorganic pigments that serve as UV filter substances.

13. The method as claimed in claim 9, wherein the average particle size of the one or more effect pigments is in the range from 50 μm to 150 μm.

14. The method as claimed in claim 9, wherein the one or more effect pigments are selected from the group consisting of pearlescent pigments.

15. The method as claimed in claim 14, wherein the one or more effect pigments are selected from the group consisting of natural pearlescent pigments.

16. The method as claimed in claim 15, wherein the one or more effect pigments are selected from the group consisting of pearl essence and mother of pearl.

17. The method as claimed in claim 9, wherein the total amount of the one or more effect pigments is in the range from 0.1% to 30% by weight, based on the total weight of the formulation.

18. A method of assessing the amount of a cosmetic or dermatological formulation applied to the skin, comprising the steps of applying a cosmetic or dermatological formulation that includes effect pigments having an average particle size of from 15 μm to 200 μm to the skin and, by reference to the visible effect to the skin, performing one or more of the following steps: estimating on which areas of the skin application has already taken place, determining whether a sufficient amount of the formulation has been applied and determining when renewed application of the formulation has become necessary.
